(19) United States
(12) Patent Application Publication
(10) Pub. No.: US 2025/0342829 A1
(43) Pub. Date: Nov. 6, 2025

(54) METHOD AND APPARATUS FOR IMPROVED MAN-MACHINE INTERACTIONS

(71) Applicant: Pfizer Inc., New York, NY (US)

(72) Inventors: Nancy Louise Ceffaratti, West Chester, PA (US); Yiorgos Perikles Christakis, Truro, MA (US); Michael Corbo, Flemington, NJ (US); Michelle Jenette Davitt, Pensacola, FL (US); Vicki Lynn Dubord, East Lyme, CT (US); Susannah Barrett Fitzgerald, Hingham, MA (US); Daniela Graham Guerrero, East Lyme, CT (US); Christine Elizabeth Kobryn, Old Lyme, CT (US); Subha Madhavan, Gaithersburg, MD (US); Russell Philip Orrico, Bethlehem, PA (US); Jeremy Robert Price, Westport, CT (US); Shobha Madapur Subbaramoo, San Diego, CA (US); Gerald Thomas Whaley, East Lyme, CT (US)

(73) Assignee: Pfizer Inc., New York, NY (US)

(21) Appl. No.: 19/196,858

(22) Filed: May 2, 2025

Related U.S. Application Data
(60) Provisional application No. 63/641,928, filed on May 2, 2024.

(51) Int. Cl. G10L 15/183; A61B 5/00; G10L 15/22; G10L 15/30; G16H 10/20; G16H 10/60

(52) U.S. Cl. CPC G10L 15/183; G10L 15/22; G10L 15/30; G16H 10/20; G16H 10/60; A61B 5/749

(57) ABSTRACT

An automated smart voice-interactive platform allows users to securely register with a provider and have physical conditions monitored, in place of interacting with a variety of human workers, in a fault tolerant and adaptive manner such that interactions improve with each additional user interaction.

### TECHNICAL FIELD

This disclosure generally relates to computing arrangements based on specific computational models, and in particular, it relates to procedures used during speech recognition processes involving human-machine dialogue.

### BACKGROUND

Automated processes for interacting with clients exist in various retail contexts in a limited and pre-scripted manner, such as interactive voice-response units (IVRUs) for telephonic customer service or grocery store checkout kiosks. However, in more complex and/or in-person client interactions, such as in a medical, legal, banking or governmental environment, client interaction processes are necessarily more personal, private and sensitive. As a result, attempts to automate such interactions have been historically disfavored by clients who are generally not comforted by having to use computers and fumble with cold, impersonal user interfaces for delicate matters. In various such instances, clients would naturally feel more comfortable dealing with another human. Nevertheless, a digital or virtual workforce in such environments could handle an estimated 80% or more of the repetitive human-performed interactions typically associated with manual client registration and physical interactions with clients, as well as reduce time needed to successfully complete client interactions. In the field of clinical trials, for example, use of an automated digital workforce could empower participants by providing ready access to detailed information, while creating substantial savings to the sponsor of the clinical trial (such as, but not limited to pharmaceutical or bio-tech companies, contract research organizations, government agencies, healthcare providers, academic institutions and non-profits) in the form of labor costs and human errors avoided, and the like. Rapid advancements in publicly available artificial intelligence (AI) tools are sparking interest in new avenues for automating advanced user interactions. While AI adoption is quickly becoming the new reality, there are many hurdles to achieving useful implementations in various real-world scenarios.

### BRIEF SUMMARY

In the exemplary embodiments described herein without limitation, various methods and systems for improving interactions over a computer network are presented. Such methods include, in various implementations, providing a first voice-responsive avatar for registering users with a service provider or sponsor, and a second voice-responsive avatar or the like for monitoring physical conditions of registered users, over the computer network. In various instances, interactions between users with the first voice-responsive avatar and/or the second voice-responsive avatar are analyzed by a large language model (LLM) program that monitors interactions with the users, and iteratively improves performance of the first voice-responsive avatar and the second voice-responsive avatar for subsequent users. In certain embodiments, the service is a medical examination or a clinical trial, and the registration of users comprises providing informed consent to and obtaining acknowledgement of consent from the users. In various instances, the physical conditions that are monitored include blood pressure and other like measurable vital signs without limitation and as described further herein. In some instances, the physical conditions are monitored with a device attached to each user that wirelessly or otherwise communicates data corresponding to measured vital signs relating to a physical conditional of each user over the computer network for secure storage and retrieval by a sponsor, provider or the like. In certain embodiments, a communication interface is provided to a staff member or other employee for personally interacting with the users when at least one of the first voice-responsive avatar and/or the second voice-responsive avatar are not functioning properly or experience extreme latency. In additional implementations, at least one of the first voice-responsive avatar and the second voice-responsive avatar communicate verbally with the users using a natural language model that is multi-lingual. Special provisioning of the avatars as well as bundling of human-machine interactions based on anticipated user latency reduce processing times and resulting latency in the computer network environment in various embodiments of the disclosed systems. Additional systems having suitable computing hardware responsive to programming instructions encoded on a tangible recording medium for implementing the disclosed methods herein are readily contemplated.

### BRIEF DESCRIPTION OF THE FIGURES

Further aspects of the present disclosure will be more readily appreciated upon review of the detailed description of its various embodiments, provided below, when taken in conjunction with the accompanying drawing figures (FIGs), of which:

FIG. 1 is a diagram of an exemplary computer network environment according to various embodiments;

FIG. 2 is an exemplary data flow chart for intelligent inquiry handling according to various embodiments;

FIG. 3 is an exemplary data flow chart for intelligent registration handling according to various embodiments;

FIG. 4. is an exemplary flowchart of an improved user registration process according to various embodiments;

FIG. 5 is an exemplary flowchart of a supplemental manual registration process according to various embodiments;

FIG. 6 is an exemplary flowchart of an improved physical condition monitoring process according to various embodiments;

FIG. 7 is an exemplary diagram of an improved user registration process according to various embodiments;

FIG. 8 is an exemplary diagram of an improved user registration and physical monitoring process according to various embodiments;

FIG. 9 illustrates two exemplary digital person or digital workforce avatars in accordance with the subject matter presented herein;

FIG. 10 illustrates some exemplary tasks or functions may be performed by a digital person or digital work force in accordance with the subject matter presented here;

FIG. 11 illustrates some additional exemplary tasks or functions that may be performed by a digital person or digital work force in accordance with the subject matter presented here; and

FIG. 12 illustrate yet some other additional exemplary tasks or functions that may be performed by a digital person or digital work force in accordance with the subject matter presented here.

### DETAILED DESCRIPTION

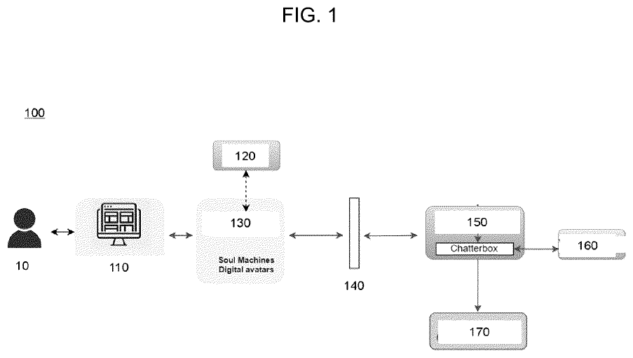

Referring now to FIGS. 1-8, wherein similar components of the present disclosure are referenced in like manner, various embodiments of methods and systems for improved human-machine interactions are disclosed.

Although a “Digital Workforce” as introduced herein will be described in the context of performing specific clinical trial tasks, including the collection of personal and medical history information and vital signs, it is readily contemplated the disclosed improvements may be used in any of a wide variety of environments.

In various embodiments, the Digital Workforce will include a first voice-responsive avatar (sometimes referred to herein as “Mia”) presented visually via a user interface (UI) on a display screen of a computing device. The Mia avatar will vocally guide participants through an initial intake, registration or consent process, and will further assist in capturing biometric and/or other secure, verifiable identification of each participant or user, such as by fingerprint capture without limitation. In various embodiments described further herein, this first avatar has an active interface with a generative AI engine that enables it to handle frequently asked question, clinical study sign-up, administration of required informed consents, high level descriptions of the purposes and goals of the clinical trial, introductions to the sponsor and its staff, answering general related questions of the participant, and providing step-by-step prompts and acknowledgements as the participants complete the registration and consent processes.

In various embodiments, the Digital Workforce will further include a second voice-responsive avatar (sometimes referred to herein as “Phillip”) that will provide instructions and confirmations to participants for collecting vital signs or other indicators of a physical condition of the participant/user, either locally or remotely. In various embodiments as contemplated and/or described further herein, this second avatar has an active interface with a generative AI engine, and/or a programmed set of stock interactions, that enables it to consistently manage clinical trial logistics, provide step-by-step instructions, answer frequently asked questions, monitor for unexpected events, direct and collect vital signs and the like, generate participant reported outcomes, collect information for clinical trial questionnaires, conduct basic differential diagnoses based on the received vital signs, record client interactions and results, and notify staff of detected emergency issues regarding a participant.

When the first and second avatars are properly configured, provisioned and implemented in a manner that prevents excessive network processing and bandwidth usage as introduced herein, many advantages are realized. These include building a dynamic corporate memory of how various participant interactions are to be properly handled, how mistakes by participants or staff are properly avoided, proper usage of protocols and data, and continuously increasing emotional awareness and intelligence with each human interaction. The disclosed systems are multilingual, mobile and rapidly scalable, and provide a holistic approach to strategically implementing a viable and accepted Digital Workforce. The improved system is implemented with various combinations of uniquely interfaced computer hardware and specially programmed software, examples of which, without limitation, will now be described in more detail hereinbelow.

An enterprise server is a powerful computer that is specifically designed to support the needs of large organizations or businesses. These servers are typically used to manage and store data, run applications, host websites, handle email services, provide access to files and resources, and facilitate communication and collaboration within the organization. Enterprise servers are characterized by their high performance, reliability, scalability, and robustness. They often incorporate advanced hardware components such as multiple processors, large amounts of random access memory (RAM), fast and robust storage systems (such as RAID arrays or SSDs), redundant power supplies, and sophisticated cooling systems to ensure continuous operation and minimized downtime. These servers are usually housed in dedicated data centers or server rooms, where they are connected to other network devices and infrastructure. They may run various operating systems, including WINDOWS SERVER, Linux, or UNIX, and can support virtualization technologies to efficiently utilize hardware resources and run multiple virtual servers on a single physical machine. Overall, enterprise servers play a critical role in the IT infrastructure of organizations, providing the computing power and resources necessary to support the various operations and functions described herein.

Enterprise servers typically consist of several key components, each playing a crucial role in the server's functionality and performance. Such components include, but are not limited to:
a Central Processor Unit (CPU) that is the “brain” of the server, responsible for executing programmed instructions and performing calculations. Enterprise servers often feature multiple CPU sockets, allowing them to accommodate multiple processors or CPU cores for enhanced performance;
RAM is used by the server to store data and instructions that are actively being processed. More RAM enables the server to handle larger workloads and run more applications simultaneously, but come with increased expense;
Data Storage for securely storing operating system files, application data, databases, and other digital assets. Storage solutions may include hard disk drives (HDDs), solid-state drives (SSDs), or storage area networks (SANs) for centralized storage management;
a motherboard that serves as the main circuit board which connects and integrates all of the server's components. It provides interfaces for connecting the CPU, RAM, storage devices, network adapters, and other peripherals;
Network Interface Cards (NICs) that enable the server to connect to a network, allowing it to communicate with other devices and access external resources such as the internet or local network services;
Power Supply Units that typically convert electrical power from the electrical outlet into power that the server's components can use (enterprise servers often feature redundant power supplies for fault tolerance and uninterrupted operation);
Since servers generate a significant amount of heat during operation, effective cooling systems are required to prevent overheating and ensure reliable performance, and such cooling systems may include one or more of fans, heatsinks, and liquid cooling solutions;
Servers often include expansion slots such as PCIe (Peripheral Component Interconnect Express) slots for installing additional expansion cards, such as RAID controllers, network adapters, or specialized accelerators for tasks like encryption or graphics processing;
an operating system (OS) such as but not limited to WINDOWS SERVER, Linux, or UNIX. The OS manages the server's resources, provides a platform for running applications, and facilitates communication with other devices on the network; and
remote management capabilities that allow staff, employees, administrators and the like to manually monitor and control the server remotely, which may include dedicated management ports, remote management software, or integrated management controllers (e.g., BMC or iDRAC).

Specific configurations and features may vary depending on the servers' intended uses, performance requirements, and budget considerations. Examples of useful enterprise servers for use as the various servers as described herein include, but are not limited to: DELL EMC POWEREDGE, HEWLETT PACKARD ENTERPRISE PROLIANT, IBM POWER SYSTEMS, CISCO UNIFIED COMPUTING SYSTEM, and LENOVO THINKSYSTEM.

The servers and user devices as described herein may communicate via a suitable computer network architecture using a wide variety of wired, wireless, passive and/or satellite connections. Some non-limiting useful network communication methods for accomplishing the functions described herein, which encompass a range of suitable technologies and protocols used for transmitting data between devices within a network or across different networks. Hardwired network communications protocols include, but are not limited to:
Ethernet is the most widely used local area network (LAN) technology for wired connections, which operates on the IEEE 802.3 standard and provides high-speed data transmission over twisted-pair copper cables or fiber optic cables;
Wireless Fidelity (Wi-Fi) is a wireless networking technology that allows devices to connect to a local area network (LAN) wirelessly, which operates on the IEEE 802.11 standard and is commonly used in homes, offices, and public spaces to provide wireless internet access;
Transmission Control Protocol/Internet Protocol (TCP/IP) is the foundational protocol suite of the internet and most computer networks, which provides reliable, connection-oriented communication between devices and facilitates the transmission of data packets across networks;
Hypertext Transfer Protocol (HTTP) and HTTP Secure (HTTPS) are application-layer protocols used for transmitting hypertext documents, such as web pages, over the internet and operates over TCP/IP, which is widely used for accessing websites and web-based services;
Domain Name System (DNS) is a hierarchical naming system used to translate human-readable domain names (e.g., www.example.com) into IP addresses, which enables, inter alia, servers and user devices to locate and communicate with other devices on the internet or within a network;
File Transfer Protocol (FTP) is a standard network protocol used for transferring files between a client and a server on a computer network. It provides a simple and reliable method for uploading, downloading, and managing files remotely;
Simple Mail Transfer Protocol (SMTP) and Post Office Protocol 3/Internet Message Access Protocol (POP3/IMAP) are email protocols used for sending and receiving emails over the internet, where SMTP is responsible for sending emails, while POP3 and IMAP are used by email clients to retrieve emails from a mail server; and
Voice over Internet Protocol (VoIP) enables voice communication over the internet or IP-based networks by converting voice signals into digital data packets for transmission and allows users to make phone calls using internet-connected devices.

In various cases, the computing devices, user devices, monitoring equipment and various servers described herein may communicate, in whole or in part, by one or more wireless data communication protocols, alternatively or in addition to hardwired communications. Suitable wireless communications protocols include, but are not limited to:

Wireless Personal Area Networks (WPAN); BLUETOOTH (a short-range wireless technology commonly used for connecting devices such as smartphones, laptops, headphones, and peripherals over short distances typically up to 10 meters that is widely used for wireless audio streaming, file sharing, and peripheral connectivity, with one example of a device or server used herein that provides BLUETOOTH mobile connectivity between devices and remaining servers being the SUMMA by Precision Digital Health (PDH)); (AIRDROP by APPLE and like protocols; Near Field Communications (NFC); Wireless Local Area Networks (WLAN); Wi-Fi (IEEE 802.11 is the most prevalent wireless networking technology for local area networks (LANs), operates over various frequency bands (e.g., 2.4 GHz and 5 GHz) and provides high-speed data transmission over relatively short distances (typically up to a few hundred feet indoors) to provide wireless internet access and network connectivity between servers and user devices, such as smartphones, tablets, laptops, and IoT devices); Wireless Metropolitan Area Networks (WMAN); WiMAX (IEEE 802.16 is a wireless broadband technology that provides high-speed internet access over a wide area, covering distances of several miles that operates on licensed or unlicensed frequency bands and is used to deliver broadband internet access to homes, businesses, and remote areas where wired infrastructure may be limited; Wireless Wide Area Network (WWAN); Cellular Networks (3G, 4G, 5G and others that provide wireless communication coverage over large geographic areas using cellular towers and base stations and enable mobile devices such as smartphones, tablets, and IoT devices to connect to the internet and communicate with each other, wherein various cellular technologies like 3G, 4G, and 5G offer increasing levels of data speed and capacity, and support a wide range of applications that may be used for the functions described herein including, but not limited to, voice calls, messaging, internet browsing, streaming media, and IoT connectivity); Satellite network connections, and Wireless Sensor Networks (WSN)) that include interconnected sensors distributed across a geographical area to monitor environmental conditions, collect data, and communicate wirelessly and are commonly used in applications such as environmental monitoring, agriculture, industrial automation, healthcare, and smart cities).

In certain embodiments, wireless communications are accomplished at least in part by ad-hoc networks, which are decentralized wireless networks formed spontaneously by wireless devices without the need for a centralized infrastructure or access points. Devices in an ad-hoc network communicate directly with each other, enabling peer-to-peer communication and collaboration. Ad-hoc networks are commonly used in scenarios where infrastructure-based networks are impractical or unavailable, such as emergency response situations, military operations, and peer-to-peer file sharing. Hybrid wired and wireless communication networks of various configurations are likewise contemplated for use.

In addition to the foregoing, some network environments herein include a virtual private network in some embodiments. A VPN, or Virtual Private Network, is a technology that allows a secure connection over the internet. It encrypts internet traffic and routes it through a remote server, hiding IP addresses and geographic location. This provides several benefits. VPNs encrypt data, making it unreadable to anyone who intercepts it, such as hackers or government agencies. This is especially important when using public Wi-Fi networks, where data is more vulnerable to interception. By hiding privacy information such as IP address, and encrypting internet traffic, VPNs prevent internet service providers (ISPs), advertisers, and websites from tracking online activities. VPNs allow access to websites and online services that may be otherwise blocked or restricted. By connecting to a server in a different country, one can bypass censorship and access content that is otherwise unavailable. VPNs also provide a certain level of anonymity by masking IP address and location, which can be useful for activities where one wants to maintain privacy, such as accessing and transmitting sensitive information. Overall, VPNs offer increased security, privacy, and freedom on the Internet and are commonly used by individuals, businesses, and organizations for various purposes, including remote access to company networks, circumventing censorship, and protecting sensitive data.

In various embodiments described herein, participants or other user types (i.e., staff and management) are described as interacting with the improved system herein using user devices. Such user devices include, but are not limited to:
Computers (Desktops, Laptops, Workstations) are primary devices used for communication over computer networks. They enable users to access network resources, exchange messages, browse the internet, and engage in various forms of communication such as email, instant messaging, video conferencing, and social media.

Smartphones and tablets are mobile devices equipped with wireless connectivity capabilities, such as Wi-Fi, BLUETOOTH and cellular networks. They allow users to access the internet, send and receive emails, make voice and video calls, send instant messages, and use a wide range of communication apps and services while on the go.

Voice over Internet Protocol (VoIP) phones are specialized devices designed for making voice calls over the internet or IP-based networks. They use VoIP technology to convert analog voice signals into digital data packets for transmission over the network. VoIP phones may be standalone devices or software-based applications installed on computers or smartphones.

Webcams and cameras are used for capturing video and images for video conferencing, live streaming, video calls, and online collaboration. They are commonly integrated into computers, laptops, smartphones, and tablets, or available as standalone devices that can be connected to a computer via USB or wirelessly.

Microphones and headsets are used for capturing voice or other audio, and transmitting corresponding audio signals for voice calls, video conferencing, online gaming, and other communication purposes. They may be built into devices such as computers, smartphones, and VoIP phones, or available as standalone peripherals that can be connected via USB or audio jacks.

Keyboards and mice are input devices used for typing text, navigating user interfaces, and interacting with software applications and communication platforms. They are essential for composing emails, instant messages, and other forms of written communication.

Displays and monitors are output devices used for viewing text, images, videos, avatars and graphical user interfaces. They are used in conjunction with computers, smartphones, and tablets to access and interact with communication apps, websites, and digital content. Displays may include speakers, or speakers may be separately provided to hear vocal information from the avatars.

Wearable devices such as smartwatches and fitness trackers may also support communication functionalities, allowing users to receive notifications, send messages, make voice calls, and access certain apps and services directly from their wrists.

In various embodiments described herein, data is communicated securely, such as by encryption. Useful encryption standards include, but are not limited to:
Advanced Encryption Standard (AES), which is a symmetric encryption algorithm widely adopted by governments and organizations worldwide. It uses block cipher encryption with key sizes of 128, 192, or 256 bits.

RRSA is an asymmetric encryption algorithm named after its inventors Rivest, Shamir, and Adleman. It's widely used for secure data transmission and digital signatures. RSA relies on the difficulty of factoring large prime numbers.

Triple DES (3DES) is a symmetric encryption algorithm that applies the Data Encryption Standard (DES) cipher algorithm three times to each data block. While it's less commonly used now due to AES's superiority, it's still present in legacy systems.

Elliptic Curve Cryptography (ECC) is an asymmetric encryption technique that relies on the algebraic structure of elliptic curves over finite fields. It offers comparable security to RSA but with smaller key sizes, making it more efficient for mobile and IoT devices.

Blowfish and TwoFish are symmetric key block ciphers designed to replace DES. Blowfish operates on 64-bit blocks and supports key sizes up to 448 bits, while TwoFish is its successor and operates on 128-bit blocks with key sizes up to 256 bits.

Diffie-Hellman Key Exchange, although not strictly an encryption algorithm, is a key exchange protocol used to establish a shared secret key between two parties over an insecure channel. It's often used in combination with symmetric encryption algorithms.

Secure Hash Algorithm (SHA) is primarily a cryptographic hash function rather than an encryption algorithm, it's crucial for ensuring data integrity and authenticity. Versions like SHA-1, SHA-256, and SHA-3 are commonly used.

Transport Layer Security (TLS) is a protocol that ensures secure communication over a computer network. It uses various encryption algorithms and cryptographic techniques to provide privacy and data integrity between communicating applications.

In various embodiments, the voice-responsive and voice-interactive avatars described herein convert speech to text for submission to a generative AI engine for comprehending the subject communication and determining a response that a human will find responsive. Generative AI textual responses are then converted back to speech for presentation to users by the avatars. Generative AI technology, particularly in the context of Natural Language Processing (NLP), has seen significant advancements in recent years. One of the most notable developments is the emergence of Large Language Models (LLMs), which have revolutionized various NLP tasks, and are readily contemplated for adaptation for use herein.

LLMs are deep learning models trained on vast amounts of text data to understand and generate human-like text. They utilize architectures such as transformers, which allow them to capture complex patterns and dependencies in language. Examples of LLMs include OPENAI's GPT (Generative Pre-trained Transformer) series (GPT-1, GPT-2, GPT-3), GOOGLE's BERT (Bidirectional Encoder Representations from Transformers), and META's ROBERTa (Robustly optimized BERT approach). LLMs have demonstrated remarkable capabilities in various NLP tasks, including text generation, text summarization, machine translation, question answering, sentiment analysis, and more. LLMs are proficient in generating coherent and contextually relevant text based on a given prompt or input. They can produce human-like responses, complete sentences, paragraphs, or even longer passages of text. Text generation applications include chatbots, virtual assistants, content creation, story generation, and code generation. LLMs can summarize long documents or articles by distilling the essential information into a shorter, more concise form. They can identify key sentences or passages and generate summaries that capture the main points of the original text. Text summarization is useful for tasks such as document summarization, news summarization, and content curation.

LLMs excel at translating text between different languages. By training on large multilingual datasets, they can learn to accurately translate text from one language to another. Machine translation applications include real-time translation services, localization of content, and cross-lingual information retrieval.

LLMs can answer questions posed in natural language by generating responses based on their understanding of the input text. They can provide relevant answers to factual questions, opinion-based questions, and more. Question answering systems are used in virtual assistants, search engines, customer support chatbots, and educational applications.

LLMs can analyze the sentiment expressed in text by identifying emotions, opinions, and attitudes conveyed by the language. They can classify text as positive, negative, or neutral and determine the overall sentiment of a piece of text. Sentiment analysis is applied in social media monitoring, customer feedback analysis, brand reputation management, market research and man-machine interface performance.

Implementations and techniques for programming useful AI engines are found in the following publications, which are incorporated herein by reference:
Vaswani, A., Shazeer, N., Parmar, N., Uszkoreit, J., Jones, L., Gomez, A. N., . . . & Polosukhin, I. (2017). Attention is All You Need. In Advances in Neural Information Processing Systems (pp. 5998-6008);
Radford, A., Wu, J., Child, R., Luan, D., Amodei, D., & Sutskever, I. (2019). Language Models are Unsupervised Multitask Learners published by OPENAI;
Brown, T. B., Mann, B., Ryder, N., Subbiah, M., Kaplan, J., Dhariwal, P., . . . & Amodei, D. (2020). Language Models are Few-Shot Learners. In Advances in Neural Information Processing Systems (pp. 1877-1901);
Holtzman, A., Buys, J., Dušek, O., Forbes, M., Bosselut, A., & Choi, Y. (2019). The curious case of neural text degeneration. arXiv preprint arXiv:1904.09751
Chen, M., Radford, A., Child, R., Wu, J., Jun, H., Luan, D., & Sutskever, I. (2021). Generative Pre-trained Transformer 3. arXiv preprint arXiv:2105.14103;
Chen, M., Radford, A., Child, R., Wu, J., Jun, H., Luan, D., & Sutskever, I. (2021). Scaling Laws for Neural Language Models. arXiv preprint arXiv:2101.02174; and
Christiano, P. F., Leike, J., Brown, T., Martic, M., Legg, S., & Amodei, D. (2017). Deep reinforcement learning from human preferences. In Advances in Neural Information Processing Systems (pp. 4302-4310).

Various processing languages are useful for specially programming the Al and avatar functions described herein. Such languages include, but are not limited to:
PYTHON is the predominant language for developing LLMs and other deep learning models. It offers extensive libraries and frameworks specifically designed for machine learning and NLP, such as TENSORFLOW, PYTORCH, and HUGGING FACE's Transformers library;
TENSORFLOW and PYTORCH are popular deep learning frameworks that provide efficient implementations of neural network architectures, including transformers. They offer high-level APIs and tools for building, training, and deploying LLMs;
TENSORFLOW is an open-source deep learning framework developed by GOOGLE. It provides comprehensive support for building and training neural networks, including LLMs, with efficient computation and scalability across CPUs, GPUs, and TPUs (Tensor Processing Units)(note: TENSORFLOW'S ecosystem includes TENSORFLOW HUB, which offers pre-trained models and modules for NLP tasks, and TENSORFLOW EXTENDED (TFX), which provides tools for productionizing machine learning pipelines);
PYTORCH is an open-source deep learning framework developed by META. It is known for its flexibility, dynamic computation graph, and intuitive interface, making it popular among researchers and practitioners for developing LLMs and other deep learning models (note: PYTORCH'S ecosystem includes the TRANSFORMERS library, maintained by HUGGING FACE, which provides pre-trained transformer models and utilities for fine-tuning and using LLMs for various NLP tasks);
JAVASCRIPT is used for deploying LLMs in software applications and browser-based environments using libraries such as TensorFlow.js and HUGGING FACE's TensorFlow.js wrapper enable developers to run pre-trained LLMs directly in the browser for tasks like chatbots, language translation, and text generation (note: TensorFlow.js allows developers to import TENSORFLOW models, including LLMs, and execute them using WebGL for accelerated GPU computation in web browsers); and
C++, which is commonly used for optimizing and accelerating the performance of LLMs, particularly during inference or deployment in production environments. Deep learning frameworks like TENSORFLOW and PYTORCH provide C++ APIs for integrating models into C++ applications and systems.

While PYTHON is the primary language for developing LLMs due to its rich ecosystem of libraries and frameworks, other languages like JAVASCRIPT and C++ play essential roles in deploying and optimizing LLMs for the various use cases and environments described herein. While some specialized programming instructions are provided in programming pseudo-code herein, it is to be understood that the textual descriptions of the functions herein can be readily converted to suitable programming instructions in any of the foregoing or other useful programming languages without undue experimentation.

Turning to the descriptions of the avatars described herein, such “Soul Machines” or life-like digital avatars, aim to replicate human-like interactions for use in the various embodiments herein. These avatars, embodied to resemble, without limitation, at least a male or female human head and/or face herein, also often referred to as “digital humans” in the art, are designed to engage with users in a natural and intuitive manner, primarily through vocal conversations. The avatars utilize Al technologies such as NLP, machine learning (ML), and emotional modeling to understand and respond to users' queries, emotions, facial responses and physical gestures. One third-party vendor of adaptable Soul Machines for the purposes described herein, is SOUL MACHINES LIMITED of Auckland, New Zealand.

Soul Machine avatars are integrated into various applications and platforms described herein, and can generally serve in roles such as customer service, education, healthcare, and entertainment, among others. With suitable specialized programming, they can provide personalized assistance, answer questions, offer emotional support, and even facilitate learning experiences as described with the methods and apparatus introduced herein.

The avatars achieve their life-like appearance and behavior through a combination of advanced graphics, animation, and AI techniques. Facial expressions, gestures, and speech are rendered dynamically based on the avatar's programming and refined by monitoring responses from the users. Additionally, the AI behind the avatars continually learns and improves over time, allowing them to become more adept at understanding and responding to human interactions. However, excessive usage of avatars with AI, without suitable programmed regulation, will lead to excessive processing delays and network latencies, which deficiencies are addressed and cured herein.

As previously mentioned, text-to-speech (TTS) and speech to text (STT) engines are used herein as an intermediary between the text needs of Generative Al and the voice responsiveness and vocals used by the avatars in various embodiments. In other instances, Automatic Speech Recognition (ASR) services may also be employed in place of TTS and STT functions. GOOGLE Text-to-Speech is one exemplary useful TTS service that is available predominantly on ANDROID smartphone devices, which provides natural-sounding speech synthesis using deep learning technologies. AMAZON POLLY is a cloud service that converts text into lifelike speech, which supports multiple languages and offers a variety of voices with different accents and speech styles. AMAZON TRANSCRIBE is a fully managed STT service that makes it easy to add speech-to-text capability to applications, which can handle audio files from different sources and accurately transcribe spoken words. MICROSOFT AZURE TEXT TO SPEECH is part of the AZURE COGNITIVE SERVICES suite, providing high-quality speech synthesis with customizable voice options. Finally, another useful service is IBM WATSON TEXT TO SPEECH, which converts written text into natural-sounding audio in multiple languages and with different voices and is part of the broader IBM WATSON suite of Al services.

In certain instances herein, voice analysis is used to identify information about users (such as a lingual accent), determine user emotions, and other information from tone and the like rather than the content of verbal communications alone, in order to correct and improve performance of the avatars in interactions with participants or the like. In such embodiments, useful voice analysis software adaptable for use include, but are not limited to:
VOICESENSE software analyzes voice patterns to provide insights into emotional states, personality traits, and more;
BEYOND VERBAL specializes in emotion detection through voice analysis, providing insights into a speaker's mood, temperament, and emotional state;
CORTI focuses on providing real-time voice analysis during phone calls to aid in medical emergencies, detecting signs of cardiac arrest, and other critical events; and
VOICEBASE offers speech analytics solutions for businesses, including transcription, keyword spotting, and sentiment analysis.

In the various embodiments described herein, one or more physical conditions of a participant, or other user, is measured during interactions with one or more of the avatars in the improved processes herein. Such physical conditions, measured as vital signs, include, but are not limited to:
Body Temperature, which is often measured using a thermometer, either orally, rectally, tympanically (in the ear), or axillary (under the arm);
Blood Pressure, which is measured using an analog or digital sphygmomanometer sometimes in conjunction with a stethoscope, provides information about the force of blood against the walls of arteries (where one non-limiting example of a wireless blood pressure monitoring device for use herein is OMRON's Bluetooth Blood Pressure Cuff);
Pulse Rate (Heart Rate) indicates the number of times the heart beats per minute (bpm) and is usually measured by a pulse meter placed at the wrist or neck;
Respiratory Rate measures the number of breaths a person takes per minute (bpm) and can be counted by measuring chest movements or breaths;
Oxygen Saturation (O2 Sat) measures the percentage of oxygen bound to hemoglobin in the blood and is often measured using a pulse oximeter, which is commonly a small device clipped onto a finger; and
a heart monitor works by detecting the electrical signals produced by the heart using electrodes, amplifying and filtering these signals, and then displaying them as an electrocardiogram (EKG) for analysis by healthcare professionals.

In some embodiments described herein, vital signs are collected by a DINAMAP vital signs monitor produced by GENERAL ELECTRIC, although other similar devices for other physical condition monitoring are readily contemplated.

The foregoing computer hardware and software can be provided through one or more specially programmed servers, such as standalone computers or enterprise servers, and arranged in a wide variety of useful implementations other than in the particular examples employed herein. Furthermore, each of the servers herein may be operated by a single entity, or in other anticipated embodiments, one or more of the servers may be operated by an independent third-party and interfaced with the appropriate protocols as described herein over a data communications network such as the Internet. One example of a useful integration platform for the improved methods described herein is MULESOFT for external integration of third party servers into the network environment. The function associated herein may be divided among many cooperating services to accommodate user scale. Likewise, it is readily contemplated that the separate functions of one or more servers as described herein can be combined into a single operating server in various instances.

Turning now to FIG. 1, therein is depicted one contemplated version of a computer network environment 100 in which the improved methods and systems introduced herein are performed, according to various embodiments of the present disclosure. In one exemplary implementation provided herein, the computer network environment 100 includes one or more users having a user device 10 for accomplishing network communications with a User Interface (UI) server 110 that operates a user interface between the user and the avatars for frontend user experience (UX).

The UI server 110 is in operative communication with a Digital Avatar server 130 that is provided for performing the processing of computer instructions necessary to operate, create and host one or more digital avatars. The Digital Avatar server 130 thus provides the “body and soul” of the “digital humans,” including their personality, language and accent recognition, animation, and sentiment analysis.

An Automatic Speech Recognition/Text-to-Speech (ASR/TTS) server 120 is provided to convert text to speech and speech to text between the Digital Avatar server 130 and a Generative AI Dynamic Response server 160, as introduced below, for the purposes described herein.

In various embodiments, a network firewall 140 is provided in the computer network environment 100 between internal and external servers of the clinical trial sponsor or other controlling entity. A network firewall is a security device or software that monitors and controls incoming and outgoing network traffic based on predetermined security rules. It acts as a barrier between a trusted internal network and untrusted external networks, such as the internet. Firewalls can be implemented as hardware appliances, software programs, or a combination of both. The primary purpose of a firewall is to protect the network and its resources from unauthorized access, malicious activities, and potential security threats. It achieves this by inspecting all incoming and outgoing traffic packets and enforcing rules to either allow or block them based on various criteria such as IP addresses, port numbers, protocols, and packet contents.

Firewalls can be configured to perform different types of filtering, including:
Packet Filtering, which examines individual packets of data and makes decisions to allow or block them based on predefined rules;
Stateful Inspection, which tracks the state of active connections and makes decisions based on the context of the traffic flow, thereby providing more robust security;
Proxy Service acts as an intermediary between internal and external networks, inspecting traffic at the application layer and providing additional security features like content filtering and caching; and
Next-Generation Firewall (NGFW) combines traditional firewall functionalities with advanced security features such as intrusion detection and prevention, application awareness, and deep packet inspection.

A Conversation Design Platform server 150 is provided in the network environment 100 to coordinate the data inputs and output of al the devices and servers in the network environment 100. This internal platform is the brain behind coordination of the digital avatars and related data processing. It further provides NLP capability and customized integrations with various platforms including Soul Machines, Generative AI inputs and outputs, and secure data storage among the respective responsible servers. NLP providers build the conversation designs for the avatars, including what they say (and do not say), how they process information, and the tasks described herein, all of which are coded in and then executed by the server 150.

A Generative AI Dynamic Response server 160 is in communication with the server 150 for receiving transcribe vocal inputs from users, identifying and generating a response using LLM processing, and transmitting the response to the server 150 for conversion to voice and presentation to the users via the avatars. In various embodiments, this server 160 processes responses from users that are necessary to complete an industry-standard Informed Consent Document (ICD) of the type commonly used in clinical trials.

All information processed via the computer network environment 100, including conversation logs with participants and the like, is stored in a Secure Storage server 170. Non-limiting examples of useful data storage servers are those produced and marketed by GNOSIS.

Other useful elements of the computer network environment 100 are introduced later herein, and various alternatives to such exemplary elements are readily contemplated.

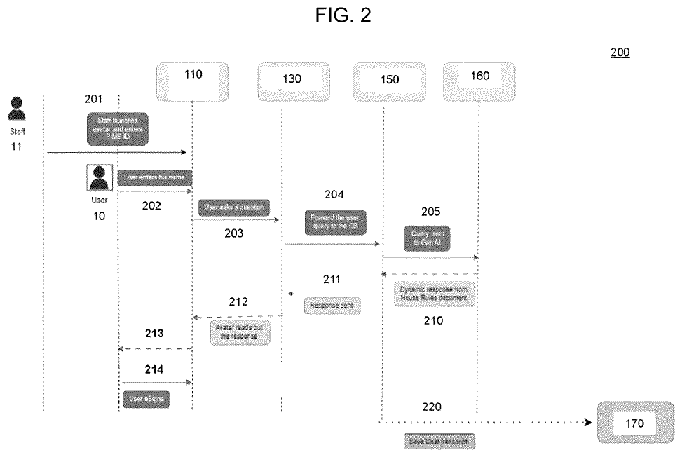

Referring now to FIG. 2, therein is depicted an exemplary data flow chart for an intelligent inquiry handling process 200 between the servers 110, 130, 150, 160 and 170, a user device 10, and a staff device 11, according to various embodiments. The process 200 commences when a staff member supporting a clinical trial or the like, using a staff device 11 initiates a first avatar that is designated for registering a user (operation 201). Separately, or after operation 201, the user 10 initiates contact with the first avatar by asking or answering a question or providing a spoken comment (operation 202). Next, the vocal user input is transmitted from the UI server 110 to the server 130 for voice to text conversation (i.e. by using the server 120) (operation 203). The user input is then forwarded to Conversation Engine 150 (operation 204), which forwards it to the Dynamic Response server 160 (operation 205). Next at operation 210, a dynamic response generated by server 160 is transmitted to server 150, which forwards the response to the server 130 for conversion from text to speech in conjunction with the server 120 (operation 211). The server 130 then responsively initiates the first avatar to vocalize and speak corresponding vocals which is transmitted through the UI server 110 (operation 212) and presented to the user 10 (operation 213). In case of additional inquiries, the forgoing process steps of the process 200 are repeated. When there are no further queries, the user ends the registration, such as, but not limited to the user executing or digitally signing a consent form or the like (operation 214). A transcript of the interactions of each user 10 during the process 200 are transmitted for secure storage in the server 170 (operation 220).

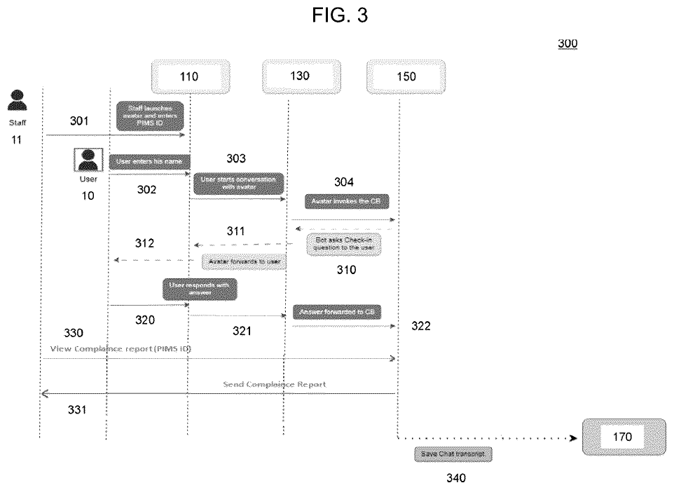

FIG. 3 is an exemplary data flow chart for an intelligent registration handling process 300 according to various embodiments. Such processes are performed, in many instances, between the servers 110, 130, 150 and 170, and the user devices 10 and staff devices 11. The process 300 commences when a staff member supporting a clinical trial or the like, using a staff device 11 initiates a first avatar that is designated for registering a user (operation 301). Separately, or after operation 301, the user 10 initiates contact with the first avatar by asking or answering a question or providing a spoken comment (operation 302). Next, the conversation started with the first avatar at the UI server 110 is transmitted to the Digital Avatar server 130 for processing (operation 303). The server 130 converts the spoken words of the user to text (as above) and forwards the converted response to the Conversation Design Platform Server 150, which, in turn prioritizes the communications based on network operating conditions and generates questions or replies as necessary via the server 160 (not shown) (operation 304). An AI-generated textual response is responsively transmitted by server 150 back to server 130 (operation 310), which in turn, initiates the avatar to perform the response vocally via the UI server 110 (operation 311), which is also optionally presented on the user device 10 when the user is remote (operation 312). In the case of a question, the user vocally responds and a recording of the vocal response is transmitted from the user device 10 to the UI Server 110 (operation 320) which is forwarded to the server 130 for text transcription (operation 321), and is then sent to the server 150 (operation 322). After completion of the user interaction herein, the staff device 11 then transmits a request for a compliance report or other confirmation of satisfactory completion to the server 150 (operation 330), which responds with the requested information (operation 331). A transcript of all the user and staff interactions during the process 300 is generated (operation 340) by the server 150 for secure storage by the server 170. In some embodiments, the first avatar only accesses generative AI algorithms that are limited to the subject matter relating to registering for the clinical trial or the like, in order to reduce processing times, network bandwidth and latency in generating responses. In such embodiments where a customer inquires on subjects completely unrelated to the clinical trial or the like, the first avatar will simply respond that it can not answer that question.

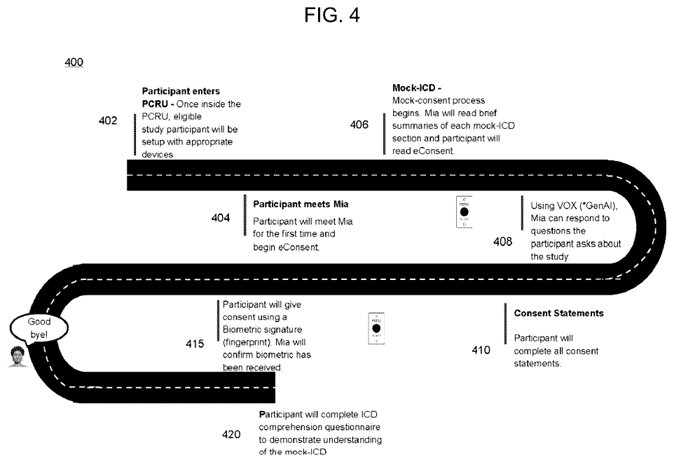

FIG. 4. is an exemplary flowchart of an improved user registration process 400 according to various embodiments. The process 400 commences when a given user 10 commences a clinical trial registration process (operation 402). In response the first avatar is activated and commences collecting information from the user necessary to complete an ICD (operation 404). Next, the consent completion is initiated by the avatar reading necessary consent information (operation 406) and soliciting one or more responses from the user (operation 408), after successful completion of which, the ICD for this user is stored (operation 410). Next, a biometric signature or the like is provided by the user (operation 415), after which additional surveys and questionnaires are presented vocally for vocal completion by the user (operation 420).

The following comprises non-limiting and exemplary pseudocode for initiating questions and answers between the avatar and the user and/or ending an inquiry, according to various embodiments:

```
    # Defining a dictionary of predefined questions and their corresponding answers
            qa_pairs = {
                    “What is your name?”: “My name is Mia.”,
                    “What time is it?”: “I'm sorry, I cannot provide real-time
                    information.”,
                    “Goodbye”: “Goodbye! Have a great day!”
            }
            def chat( ):
                    print(“Welcome! I am Mia!”)
                    print(“You can start asking questions or say ‘Goodbye’ to exit.”)
            while True:
                    user_input = input(“User: ”)
# Convert user input to lowercase for case-insensitive matching and text-to-speech
conversion:
            user_input = user_input.lower( )
            if user_input == “goodbye”:
                    print(“Mia:”, qa_pairs[“Goodbye”])
            break
            # Search for a matching question in the predefined QA pairs
                    matched_question = None
                    for question in qa_pairs:
                            if question.lower( ) in user_input:
                            matched_question = question
                    break
                            if matched_question:
                            print(“Mia:”, qa_pairs[matched_question])
                            else:
                            print(“Mia: I'm sorry, I don't understand. Please rephrase
                            your question.”)
            # Start the conversation
                    chat( )
```

From the foregoing exemplary programming code, a dictionary “QA_pairs” contains predefined questions as keys with their corresponding correct answers (and reasonably similar as determined by AI) as values. We define a function chat( ) to simulate the conversation with the user/participant. Inside this function, we continuously prompt the user for need information input until all information is collected and/or the participant vocalizes “Goodbye” (or similar definitive indication to end further interactions). We convert the vocal user input to lowercase text for case-insensitive matching and AI processing (likewise received textual AI outputs are then converted to speech for use by the avatars in interacting with participants). We search for a matching question in the predefined QA pairs. If a match is found, the avatar speaks the corresponding answer. Otherwise, the avatars inform the user that the question is not understood, or similar. When the user says “Goodbye”, each avatar will vocally reply with a farewell message and exit the conversation loop.

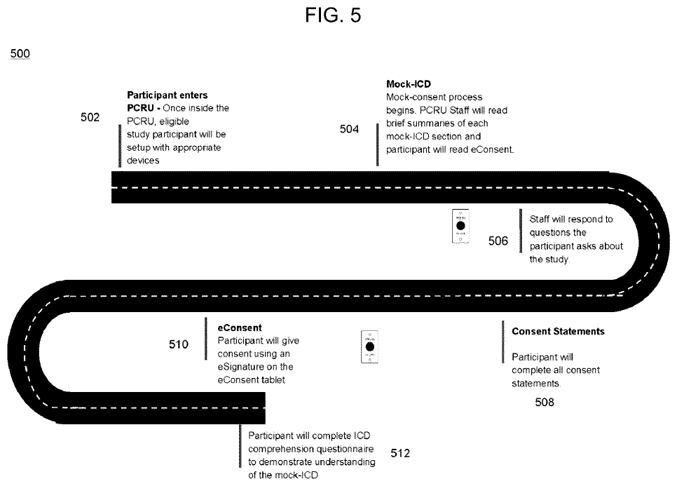

FIG. 5 is an exemplary flowchart of a supplemental manual registration process 500 according to various embodiment. This process 500, or any useful portion thereof, may be employed when an avatar is malfunctioning, unavailable or is incapable of a reply to a given inquiry in the process 400, among other examples of errors. The process 500 commences when a user 10 initiates the registration process (operation 502), after which a staff initiates the consent process (operation 504). Next, the staff asks and responds to questions (operation 506), after successful completion of which a consent form presented to the user (operation 508) and completed (operation 510). Any desired follow-up surveys and questionnaires are then presented for completion by the user via the staff, in place of the avatar (operation 512).

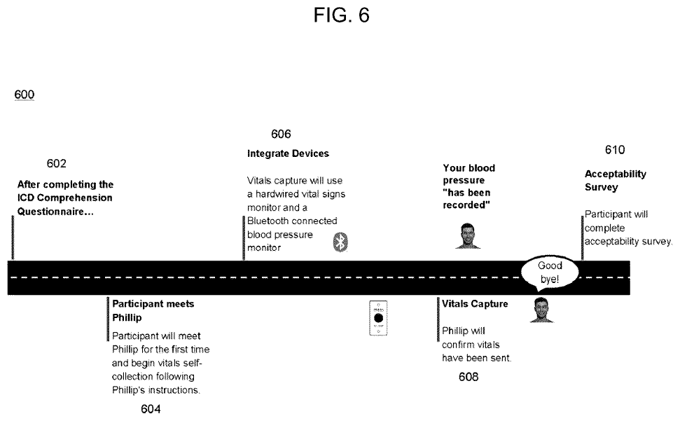

FIG. 6 is an exemplary flowchart of an improved physical condition monitoring process 600 according to various embodiments. After confirmation of completion of the registration/consent process (operation 602), a second avatar is presented and introduced (operation 604). The second avatar instructs and monitors the user while vitals are captured by a monitoring device (operation 606). Next, the system confirms receipt of vitals through the second avatar (operation 608), after which the user completes any additional surveys and questionnaires required by the clinical trial (operation 610). In some embodiments, the second avatar is interfaced with the server 160 to formulate responses and instructions to users using generative AI algorithms. In additional embodiments, the second avatar only accesses generative AI algorithms that are limited to the subject matter relating to the clinical trial or the like, in order to preserve processing, network bandwidth and latency in generating responses. In still further embodiments, the second interface does not fully transact, or in some instances, transact at all with the server 160 to formulate instructions and query responses, and instead uses a series of pre-programmed responses directed to the usual types of interactions associated with vital sign collection processes.

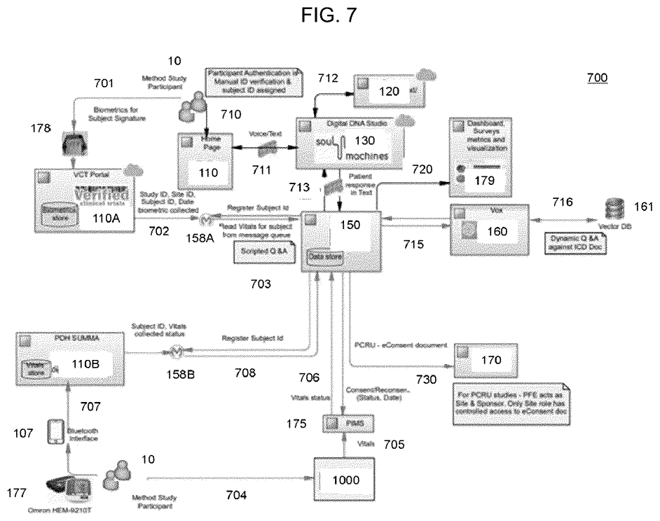

FIG. 7 is an exemplary diagram of an improved user registration process 700 according to various embodiments. The process 700 commences when a user 10 submits to a biometric scan or the like for identification and later consent affirmation by scanning a fingerprint or the like with a biometric scanning device 178 (operation 701). The biometric scan is processed and securely stored by a Clinical Trial portal server 110A which may be part of the UI Interface server 110, and the resulting data is forwarded to a Data Queue server 158A (operation 702). The server 158A then forwards the data to server 150 for routing to the Secure Storage server 170 (operation 703).

Next, the user interfaces with the first avatar via the UI server 110 for completing an improved registration process (operation 710). Vocals from the user spoken to the first avatar are recorded by the server 110 and forwarded to the Digital Avatar server 130 for handling as described in the foregoing (operation 711). The vocals are sent from the server 130 to the server 120 for conversion from speech to text (operation 712), and the converted text is then sent to the server 150 for routing (operation 713). The server 150 sends the data, alone, or bundled with other data processing requests, to the server 160 for comprehending and formulating a response to the user(s) based on ICD details or other guidelines stored for use by the server 160 in a Criteria database 161 (operation 715). Text responses and further queries generated by the server 160 are returned along the same data route for presentation to the user by the first avatar.

Once the registration process for a user has been completed, and the consent of the user recorded by biometrics or the like, the process 700 continues with the user 10 submitting to a vital sign physical collection process managed by the second avatar. In some embodiments, vital sign collection is conducted via a remote, independent Vital Sign Collection server 1000 (operation 704). The server 1000 transmits the actual vital signs to a Personal Information Management System (PIMS) server 175 for secure and private storge (operation 705). The PIMS server 175 may then provide an indication that vital signs collection was successfully completed without transmitting the private data itself outside of secure storage in order to protect the privacy of the user (operation 706). In an additional embodiment, the user may instead collect vital signs themselves, in place of the service provided by the server 1000, using a vital signs monitoring device 177 having a wireless (i.e., BLUETOOTH) interface 107. In such instances, the vital signs collected by the user 10 in conjunction with the instructions from the second avatar are transmitted to Vital signs server 110B for secure storage and an indication of successful vital signs collection is generated (operation 707). Subsequently, the indication is transmitted to a Data Queue server 158B and forwarded to the server 150 for further processing and coordinated data storage for use by the clinical trial (operation 708).

After successful completion of the registration and vital sign collection processes, the user 10 is in various instances presented with one or more questionnaires or surveys concerning the clinical trial and the improved processes used thereby, and then the responses are transmitted by the server 150 to a Survey Management server 179 for storage and data analysis (operation 720).

All the data collections referenced above as well as transcripts of conversation logs with a plurality of users is routed by the server 150 for secure storage in the databases of server 170 (operation 730).

In some embodiments, the ICD document may be broken down or segmented, or vecterized, or indexed into a plurality of portions, and stored in the Response Server 160. Vector DB 161 may be utilized to search information from the segmented/vecterized/indexed ICD document. In some other embodiments, a software module such as a Retrieval-Augmented Generation module or model may be used to facilitate semantic searches. For examples, users may ask questions using various phrases or meanings, or in different formats, and the Response Server 160, through this RAG module, may be able to break down the user's questions, subsequently pick out important or primary words or phrases, and match up with the portion or segments of data from the ICD document. Then retrieve the data, and in some cases rank the data, and pick the top ranked data to formulate an answer. In some embodiments, the ICD document may include tables, and the RAG module or model may be utilized to retrieve information from these tables to formulate a response to user's questions. In yet another embodiment, a user's question may be deemed outside the scope of the ICD document, to avoid the risk providing in-accurate information, the Response Server 160 may be configured to deny a response to the user, or to provide a non-informative response to the user, for example, a response indicating to the user that such question is not to be answered at this time.

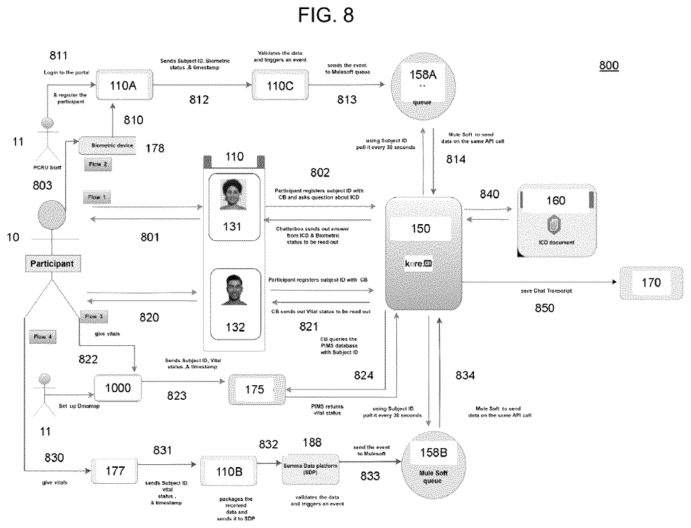

Referring now to FIG. 8, therein is depicted an exemplary diagram of a combined improved user registration and physical monitoring process 800 according to various embodiments. The process commences when a user 10 interacts with the first avatar 131 and commences a registration process (operation 801). All user vocals responsive to the first avatar 131 are locally processed by the server 110, which now additionally perform the functions previously attributed to servers 120 and 130 (operation 802). The vocals converted to text are transmitted by the server 110 to Conversation Design Platform server 150 that coordinates user interactions with the Generative AI Dynamic Response server 160 as in the foregoing manners. Next, the user 10 submits to biometric scan in accordance with instruction isssued by the first avatar 131 by using the biometric device 178 for identification and/or consent acknowledgement (operation 803).

Alone, or with the help of a local or remotely located staff 11 (operation 811), the user biometrics are transmitted to and processed by the server 110A for verification, and when successfully completed, a validation signal is sent to a Validation Platform server 110C for recording (operation 812), The validation is then placed in the Queue server 158A (operation 813) for transmission to and processing by the server 150 (operation 814). The first avatar 131 will then issue vocal confirmation that the biometrics have been recorded to the user or, if there was an error in any of the foregoing operations, the first avatar 131 will speak instructions for correcting the error in various embodiments. Other inquiries required for completing the consent and registration processes of the clinical trial are also performed via the first avatar 131 in similar manner to the foregoing descriptions.

After the registration of a user is complete and consent is obtained, the user next interacts with the second avatar 132 that manages the collection of vital signs from the user (operation 820), and the interactions are monitored and coordinated by the server 150 (operation 821). In certain embodiments, the user then has vital signs measured by an operator of the Vital Sign Collection server 1000 (operation 822). The user ID of the user, the measured vital signs and a timestamp representing the time of data collection are sent to the PIMS server 175 for secure and private storage (operation 823). An indication and verification of the correct collection of vital signs data (including user ID and timestamp) is then transmitted to the server 150 (operation 824).

In some embodiments, the user 10 instead measures vital signs data themselves using a vital sign monitoring device 177 (operation 830), which data is then transmitted by BLUETOOTH or the like to the server 110B for useful data packaging (operation 831). The server 110B then transmits the packaged data to Data Platform server 188 (operation 832), which in turn, transmits the data for placement in the data queue maintained by server 158B (operation 833). The server 158B then provides the data to the server 150 for utilization with the clinical trial processes described herein (operation 834).

All verbal interactions between users and the avatars are transmitted for processing by the Generative AI Dynamic Response server 160 (operation 840). After the collection of data is complete for each of the plurality of users/participants in the clinical trial, each generated ICD and interaction and conversation transcription logs are securely stored in server 170 (operation 850).

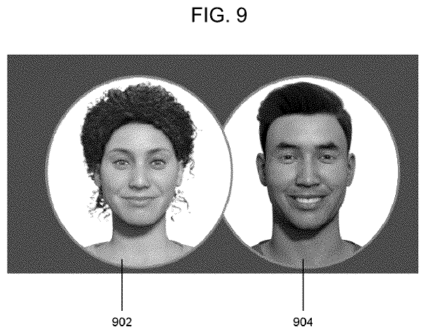

Referring now to FIG. 9, where two digital person or digital work force avatars are presented here for illustration purposes. Digital person or digital work force avatar 902 may have features that resemble a human female and digital person or digital work force avatar 904 may have features that resemble a human male. It should be appreciated that in some embodiments, the functionalities or tasks performed by the two avatars are interchangeable depending on the specific needs. Furthermore, the features associated with the avatars may be freely modified by a designer.

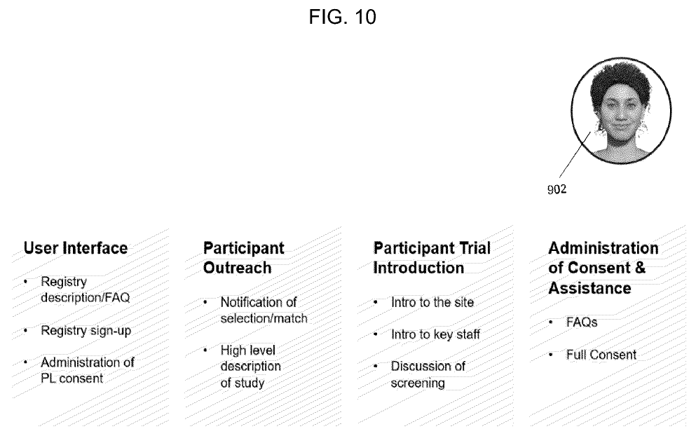

In some embodiments, the digital person or digital work force avatar 902 may be configured to perform tasks such as administration of consent and assistance for clinical trial applications, as illustrated in FIG. 10, where the avatar 902 may be configured to answer frequently asked questions (FAQs) and take in consents from applicants or patients. In some embodiments, an user interface may be utilized for registry descriptions or FAQs, registry sign-up, or administration of consents. The avatar 902 may also be tasked with clinical trial participant outreach tasks such as notification of selection/math, and/or high-level description of study. In yet another embodiment, participant trial introductions tasks such as making introductions to an informational site, introductions to key staff members, or discussion of screening may also be performed by digital work force avatar 902.

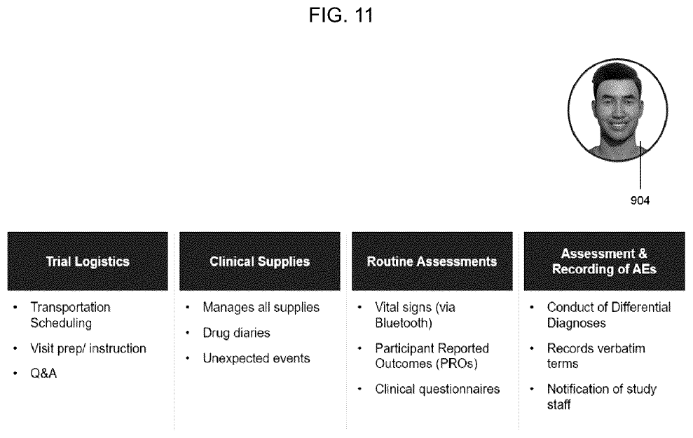

In yet another embodiment, referring now to FIG. 11, digital work force avatar 904 may be configured to perform assessment tasks such as vital sign measurements (e.g., using Bluetooth devices), participant reported outcomes (PROs), or clinical questionaries. Digital work force avatar 904 may also be tasked with trial logistical functions such as transportation scheduling, visit prep/instruction, and/or question and answer sessions. In some other embodiments, digital work force avatar 904 may also perform clinical supply tasks such as managing supplies, record drug diaries and/or unexpected events. In yet another embodiment, digital work force avatar 904 may be configured to perform assessment and recording functions such as conducting differential diagnoses, recording verbatim terms and/or notifying study staffs.

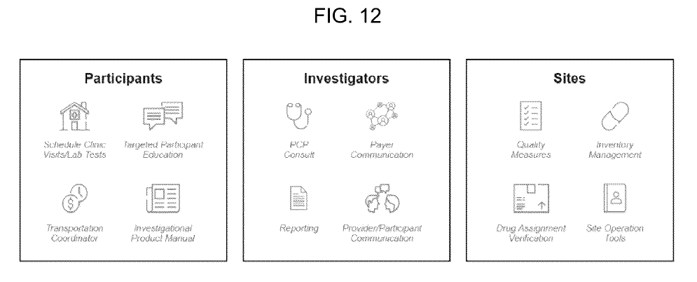

Furthermore, referring now to FIG. 12, a digital person or digital work force may be configured to perform clinical trial participant related tasks such as scheduling clinical visits and/or lab tests, educating targeted participants, coordinating transportations, or providing product manuals. In some embodiments, the digital person or digital work force may be further configured to assist investigators with PCP consultation, payer communication, reporting, or communications to and between providers and participants. In yet another embodiment, a digital person or digital work force may be configured to assist with site management functions such as quality measurements, inventory managements, drug assignment verifications, or site operation tools.

Various technical hurdles to implementation have been encountered and overcome by the improvements introduced herein. Network latencies are a particular concern when seeking to provide useful human-machine interfaces, as humans will quickly become frustrated with delays in avatar response in the same manner as with incorrect responses therefrom. Network latencies are particularly prevalent with the generative AI determinations of user comments and questions, the conversions of the same and the determined avatar responses between text and speech. In addition, the generation of avatar vocals, the deciphering of user replies to the avatars, and the generation of virtual head and facial movements of the avatars by the Digital Avatar servers further require significant processing time in each instance, which builds over many dozens, hundreds or thousands of simultaneous users, thus contributing further to network latencies. In order to improve user interactions while improving network latency performance, several technological solutions have been employed.

Whenever the avatars fail to operate properly, a process for quickly introducing staff to take over the registration or vital monitoring processes from the avatars are readily provided. The server 150 continuously measures latencies in the various user interactions as they proceed and when latencies greater than approximately five seconds, or other useful value, an automatic prompt is generated to cause the avatar to acknowledge the delay and assure that a response is forthcoming. When the latency level takes significantly longer than the threshold, the interaction may be interrupted, and the avatars may prompt the user to restart the most recent interaction. In various embodiments, the generative AI used by the avatars is impoverished to include only those topics that are likely to be encountered during a specific clinical trial and will not attempt to apply unrelated questions or other prompts. For standard and predictable portions of the registration and physical condition monitoring processes, generative AI may not be used at all, and a pre-programmed script of the most common interactions is used instead by one or both of the avatars 131, 132 during particular portions of such processes, as contemplated in various embodiments.

Human induced delays can also occur, such as when a user is trying to operate the biometric or vital sign monitoring devices or when trying to think of an answer to an inquiry. The server 150 monitors when such human induced latencies typically occur during the registration and vital signs monitoring processes, and responsively learns to bundle data from among many simultaneous users and the like for processing during the times when a given process or step thereof is in such identified human latency periods. In such manner, the processing which require delays are batch processed during such human-induced latency periods to increase overall system response times during human interactions.

Generative AI systems learn to identify and correct occurrences of errors in past interactions with humans and avoid them in the future by modifying the voice prompts the avatars give to users over time. Such errors as encountered during implementation include user name mispronunciation, inability to properly interpret voice accents and tone of voice, incorrect collection of vitals by users, use of incorrect vocabulary or colloquialisms by users that “confuses” AI, and users incorrectly exiting a process at a given time. Participants using the improved man-machine interfaces described herein include ease of use, absence of “tech-related anxiety” and increased confidence during the participation in the clinical studies or trials.

Although the best methodologies have been particularly described in the foregoing disclosure, it is to be understood that such descriptions have been provided for purposes of illustration only, and that other variations both in form and in detail can be made thereupon by those skilled in the art without departing from the spirit and scope thereof, which is defined first and foremost by the appended claims.

What is claimed is:

1. A method for improving interactions over a computer network, comprising:
providing a first voice-responsive avatar for registering users with a service over the computer network;
providing a second voice-responsive avatar for monitoring physical conditions of the users registered with the service over the computer network; and
storing interactions of users with the first voice-responsive avatar and the second voice-responsive avatar in a database used by a large language model (LLM) program to analyze interactions with the users, and to iteratively improve performance of the first voice-responsive avatar and the second voice-responsive avatar for subsequent users, wherein the first voice-responsive avatar and the second voice-responsive avatar are provisioned to reduce network latency.

2. The method of claim 1 wherein the service is a clinical study.

3. The method of claim 1 wherein registration of users comprises obtaining consent of the users.

4. The method of claim 1 wherein the physical condition comprises at least one of a blood pressure, a respiratory rate, a body temperature, a pulse rate, a blood oxygenation level and a heart condition of the user.

5. The method of claim 1, further comprising monitoring the physical condition with a device attached to each user that wirelessly communicates vital signs corresponding to the physical conditional over the computer network for secure storage and retrieval.

6. The method of claim 1, further comprising providing a communication interface to a staff of the service for interacting with the users when at least one of the first voice-responsive avatar and the second voice-responsive avatar are not functioning.

7. The method of claim 1, wherein at least one of the first voice-responsive avatar and the second voice-responsive avatar communicate verbally with the users using a natural language model that is multi-lingual.
